(19) United States
(12) Patent Application Publication
(10) Pub. No.: US 2008/0213172 A1
(43) Pub. Date: Sep. 4, 2008

(54) RADIOIMAGING MOIETIES COUPLED TO PEPTIDEASE-BINDING MOIETIES FOR IMAGING TISSUES AND ORGANS THAT EXPRESS PEPTIDASES

(75) Inventors: J. W. Babich, Cambridge, MA (US); W. C. Eckelman, Cambridge, MA (US); F. J. Femia, Cambridge, MA (US); Craig Zimmerman, Cambridge, MA (US)

Correspondence Address: FOLEY AND LARDNER LLP;SUITE 500, 3000 K STREET NW, WASHINGTON, DC, 20007, US

(73) Assignee: Molecular Insight Pharmaceuticals, Inc.

(21) Appl. No.: 11/847,276

(22) Filed: Aug. 29, 2007

Related U.S. Application Data
(60) Provisional application No. 60/823,884, filed on Aug. 29, 2006.

(51) Int. Cl. A61K 51/00; C07F 13/00; A61P 43/00

(52) U.S. Cl. 424/1.61; 424/1.65; 534/14

(57) ABSTRACT

Conjugates, methods and kits are described for imaging tissues and organs that express one or more peptidases. In a preferred embodiment of the invention, a series of di-(2-pyridylmethyl)amine (D) ligands, which can bind M(CO)₃⁺ [M=Tc or Re], were coupled to lisinopril (L). Aliphatic tethers with varying number of methylene groups (3, 4, 5, and 7; D(C₄)L, D(C₅)L, D(C₆)L, and D(C₈)L, respectively) were utilized, with in vitro inhibitory activity increasing with increasing number of methylene groups. The D(C₈)L conjugate was observed to be significantly more potent than D(C₄)L. In vivo specificity for ACE was studied in both tissue distribution and gamma imaging studies, demonstrating localization in tissues with high ACE content. Localization was blocked by pretreatment with lisinopril.

### CROSS-REFERENCE TO RELATED APPLICATIONS

This application claims priority to U.S. Provisional Application Ser. No. 60/823,884 filed Aug. 29, 2006, the disclosure of which is incorporated herein by reference in its entirety.

### ACKNOWLEDGEMENTS

This work was supported by a grant from the National Institute of Health (NIH), Department of Health and Human Services, 1-R43-HL075918-01. The federal government may have certain rights in the invention.

### INTRODUCTION

A variety of tissues (including blood) and organs express varying levels of peptidases (also termed proteases, proteinases and proteolytic enzymes). Expression levels may vary also depending on a pathological condition (or absence thereof) associated with a tissue or organ. For example, it is known that high levels of angiotensin-converting enzyme (ACE) are found in the myocardium of heart failure victims.

The MEROPS database (http://merops.sanger.ac.uk/) is an information resource for peptidases and the proteins that inhibit them. The MEROPS database also contains a long listing of small molecule inhibitors of selected peptidases. See, Rawlings, N. D., Morton, F. R. & Barrett, A. J. (2006) MEROPS: the peptidase database. Nucleic Acids Res 34, D270-D272. The contents of this database, particularly release 7.50, are incorporated into this specification by reference herein.

As inhibitors of peptidases, these molecules (whether macromolecules, like proteins, or small molecules, including peptides and existing drugs or drug candidates) also bind to the peptides that they inhibit with a certain affinity.

ACE, An Exemylary Peptidase Despite the trend of decreasing death rates attributable to ischemic heart disease and stroke, the prevalence of congestive heart failure and the resultant death rates in the United States have almost tripled over the past three decades. See, S. Y. Chai, F. A. O. Mendelsohn, G. Paxinos, Neuroscience, 20: 615-627 (1987). It is estimated that over the next two decades, heart failure due to coronary heart disease will surpass all infectious diseases to become the leading cause of death in the world. See, M. R. Cowie, D. A. Wood, A. J. S Coats, S. G. Thompson, P. A. Poole-Wilson, V. Suresh, G. C. Sutton, Eur. Heart J, 20: 421-428 (1999).

Hence, a need exists for newer and better ways to diagnose, treat and monitor the progression of certain diseases, such as heart failure.

### Lisinopril, An Exemplary Peptidase-Binding Moiety

Lisinopril, a clinically utilized ACE inhibitor for the treatment of hypertension and congestive heart failure, has been shown to cause direct inhibition of ACE. Based upon preliminary autoradiography results from heart slices of patients with congestive heart failure, See, V. Dilsizian, J. Shirani, Y. H-C. Lee, D. Kiesewetter, E. M. Jagoda, M. L. Loredo, W. C. Eckelman, Circulation, 104:17, 3276 (2001), the inventors believe that ACE may be an attractive molecular target for the diagnosis and staging of heart failure as well as response to therapy. Analogously, the inventors believe that the over-expression of other peptidases in certain tissues and organs can be exploited to diagnose, treat and monitor the progression of a wide variety of pathological conditions. Such pathological conditions include, but are not limited to, heart failure, cardiomyopathy, lung disease, kidney dysfimction, renal failure, inflammation, atherosclerosis, vulnerable arterial plaques or neoplasms, such as breast cancer, prostate cancer, gastric cancer, hepatocellular carcinoma, lung cancer and the like. Still other pathological conditions include cardiovascular diseases, in general, including diabetic nephropathy, excess tissue ACE activity, chronic renal failure due to non-insulin-dependent diabetes mellitus or hypertension, hypertensive peripheral vascular disease, emphysema (or chronic obstructive pulmonary disease—COPD), and the like.

### SUMMARY OF THE INVENTION

The present invention relates to a series of conjugates which combine peptidase-binding moieties (such as substances that inhibit peptidases) with radiopharmaceutical moieties (including radiotherapeutic and radio-imaging moieties) or optical imaging moieties. Peptidases include but are not limited to exopeptidases, such as carboxypeptidases and aminopeptidases, and endopeptidases, such as serine-, cysteine-, aspartic- and metalloendopeptidases. A “moiety” is a molecule that can exist independently of another moiety. Hence, mere substituents (i.e., functional groups), like hydroxyl, halide and the like, are not “moieties” within the meaning of this invention.

In a specific embodiment of the invention, a series of conjugates based on the coupling of a metal chelate complex and lisinopril, an inhibitor of dipeptidyl carboxypeptidase (a.k.a. angiotensin-converting enzyme), is described. Hence, a series of lisinopril-based ligands (described in further detail below), which are capable of binding metallic species, e.g., a M(CO)₃⁺ [M=Tc or Re, especially non-radioactive and radioisotopes thereof] core, are synthesized and evaluated. Examples of suitable ligands include, but are not limited to, di-(2-pyridyhnethylene)amine, di-(2-quinolinemethylene)amine, di-(2-isoquinoline)amine, and the like, which are coupled to lisinopril or other peptidase-binding moiety via, for example, an aliphatic tether. In vitro analyses demonstrate that increasing the number of methylene groups contained in an aliphatic tether results in an increase in inhibitory potency. In vivo specificity for ACE is also studied in the presence or absence of free lisinopril using normal rats. These in vivo studies demonstrate localization of radiotracer in tissues with high ACE content, which localization is blocked by pretreatment with free lisinopril.

In another embodiment of the invention, the preparation of a novel series of ^{99m}Tc-labeled ACE inhibitors is described. These conjugates have the potential to monitor ACE expression in vivo and could be useful, e.g., in the staging of cardiovascular disease, especially congestive heart failure. Surprisingly, the most potent compound in this series, ^{99m}Tc-D(C₈)L, is the one bearing the longest tether. This conjugate is evaluated in animal models of ACE over-expression with the goal of assessing its ability to, for example, diagnose and stage heart failure (e.g., by quantifying the expression of ACE in the myocardium). Accordingly, a method of imaging a tissue or organ that expresses ACE is one application of the invention. In the particular case of ACE expression, a method of imaging lung tissue, kidney tissue, hear tissue, tumor tissue or combinations thereof is disclosed.

The invention is also directed to optical (e.g., fluorescence, chemiluminescence or phosphorescence) imaging moieties coupled to peptidase-binding moieties, for example, non-radioactive (i.e., “cold”) rhenium chelate complexes using di-(2-quinolinemethylene)amine or di-(2-isoquinoline)amine as a chelating ligand tethered to a peptidase-binding moiety. Examples of applications of optical imaging are disclosed in Wei L, Babich J W, Ouellette W, Zubieta J., Developing the {M(CO)3}+ core for fluorescence applications: Rhenium tricarbonyl core complexes with benzimidazole, quinoline, and tryptophan derivatives. Inorg Chem.2006 Apr. 3; 45(7):2006 3057-66 and James S, Maresca K P, Babich J W, Valliant J F, Doering L, Zubieta J., Isostructural Re and 99mTc complexes of biotin derivatives for fluorescence and radioimaging studies. Bioconjug Chem. 2006 May-June; 17(3):590-6. The invention also encompasses radiotherapeutic moieties as a coupling partner for a peptidase-binding moiety. The term “radiopharmaceutical moiety” is meant to encompass a radio-imaging moiety, a radio-therapeutic moiety or both. An example of a radio-therapeutic moiety might be a rhenium-186 or rhenium-188 tri(carbonyl) di-(2-pyridylmethylene)amine chelate complex.

### BRIEF DESCRIPTION OF THE FIGURES

FIG. 1 shows a synthetic scheme for the preparation of di-(2-pyridylmethyl)amine (D) chelates coupled to lisinopril (L).

FIG. 2 illustrates dose curves of Lisinopril and D(Xₓ)L compounds in an in vitro biochemical assay.

FIG. 3 shows tissue distribution of ^{99m}Tc-D(C₅)L in normal and lisinopril-pretreated (1 mg/kg, i.v.) Sprague Dawley rats at 15 minutes.

FIG. 4 shows radiographic images of ^{99m}Tc-D(C₅)L in Sprague Dawley Rats (Left panel: not pretreated with lisinopril; Right panel: pretreated with lisinopril).

FIG. 5 shows ligands and corresponding ligand-metal complexes. The ligands and ligand-metal complexes can be conjugated to either the C-terminal or the N-terminal of a peptide sequence.

FIG. 6 shows ligands and corresponding ligand-metal complexes for attachment to an amino functionality.

FIG. 7 shows ligands and corresponding ligand-metal complexes for attachment to carboxy functionality.

FIG. 8 shows a synthetic scheme of a compound of the present invention including a chelation step.

FIG. 9 is an anterior view of whole-body planar images show in vivo distribution in control (A) and lisinopril-pretreated (B) rats at 10 minutes after injection of ^{99m}Tc(CO)₃D(C₈)L (MIP-1037).

FIG. 10 shows Small Animal SPECT/CT Images show lung activity in the control rat (A) after injection of ^{99m}Tc(CO)₃D(C₈)L (MIP-1037) which is not present in the rat pretreated with lisinopril (B).

FIG. 11 shows the results of Table II in a bar chart.

### DETAILED DESCRIPTION OF THE PREFERRED EMBODIMENTS

In a preferred embodiment of the invention, probes for imaging ACE expression are prepared. Lisinopril (“L”), an inhibitor of ACE, was used as the starting pharmacological motif. Di-(2-pyridylmethyl)amine (“D”), a ligand capable of binding M(CO)₃⁺ [M=Tc or Re], is incorporated into lisinopril by amide bond formation at the ε-amine of the lysine residue of lisinopril. The ligands were equipped with aliphatic tethers containing varying number of methylene spacer groups (3, 4, 5, and 7; designated D(C₄)L, D(C₅)L, D(C₆)L, and D(C₈)L, respectively). See, FIG. 1 herewith.

ACE inhibition was evaluated in vitro against rabbit lung ACE using a colorimetric assay. In vivo specificity for ACE was determined for ^{99m}Tc-D(C₅)L by studying tissue distribution and clearance in the presence (n=6/time point) or absence (n=4/time point) of lisinopril (1 mg/kg i.v.) using normal male Sprague Dawley rats at 15, 60, and 120 minutes post-injection.

### EXAMPLES

The contents of all reference citations mentioned in the specification are incorporated by reference herein.

### Preparation of Conjugates

Lisinopril was obtained from LKT Laboratories (Saint Paul, Minn.). All ligands were synthesized according to published literature procedures with slight modifications. See, M. K. Levadala, S. R. Banerjee, K. P. Maresca, J. W. Babich, J. Zubieta, Synthesis, 11: 1759-1766 (2004); L. Wei, J. Babich, W. C. Eckelman, J. Zubieta, Inorg. Chem., 44: 2198-2209 (2005). Elemental analysis was performed by Desert Analytics (Tucson, Ariz.) and electrospray mass spectrometry by HT Laboratories (San Diego, Calif.).

D(C₄)L (1): Yield=40% (0.68 g). ¹H NMR (CDCl₃, ppm): 8.50 (m, 2H), 7.62 (m 2H), 7.43 (m, 2H), 7.13 (m, 8H), 3.85 (m, 4H), 3.69-2.60 (mm, 11H), 2.26-1.41 (mm, 16H). MS(ESI): m/z 674 (M⁺+1), m/z 672 (M⁻−1). Anal. Calcd. for C₃₇H₄₈N₆O₆.1.5H₂O: C, 63.50; H, 7.35; N, 12.01; O, 17.15. Found: C,63.44; H, 7.11; N, 12.24, O, 17.17. (MIP-1039)

D(C₅)L (2): Yield=34% (0.61 g). ¹H NMR (CDCl₃, ppm): 8.51 (m, 2H), 7.65 (m 2H), 7.51 (m, 2H), 7.13 (m, 8H), 3.92 (d, 4H), 3.69-2.65 (mm, 11 H), 2.27-1.46 (mm, 18H). MS(ESI): m/z 688 (M⁺+1), m/z 686 (M⁻−1). Anal. Calcd. for C₃₈H₅₀N₆O₆.H₂O: C, 64.75; H, 7.44; N, 11.92; O, 15.89. Found: C, 64.77; H, 7.35; N, 11.92; O, 16.07. (MIP-1003)

D(C₆)L (3): Yield=13% (0.23 g). ¹H NMR (CDCl₃, ppm): 8.50 (m, 2H), 7.64 (m 2H), 7.49 (m, 2H), 7.15 (m, 8H), 3.86 (d, 4H), 3.68-2.60 (mm, 11H), 2.26-1.41 (mm, 20H). MS(ESI): m/z 702 (M⁺+1), m/z 700 (M⁻−1). Anal. Calcd. for: C₃₉H₅₂N₆O₆.2.5 H₂O: C, 62.80; H, 7.70; N, 11.27; O, 18.23. Found: C, 62.82; H, 7.47; N, 11.40; O, 17.91.

D(C₈)L (4): Yield=35% (0.57 g). ¹H NMR (CDCl₃, ppm): 8.50 (d, 2H), 7.63 (m 2H), 7.50 (m, 2H), 7.13 (m, 8H), 3.85 (d, 4H), 3.69-2.53 (mm, 11H), 2.23-1.22 (mm, 24H). MS(ESI): m/z 730 (M⁺+1), m/z 728 (M⁻−1). Anal. Calcd. for: C₄₁H₅₆N₆O₆.H₂O: C, 65.93; H, 7.83; N, 11.25; O, 14.99. Found: C, 65.64; H, 8.21; N, 11.20; O, 14.48. (MIP-1037)

### In Vitro Analysis

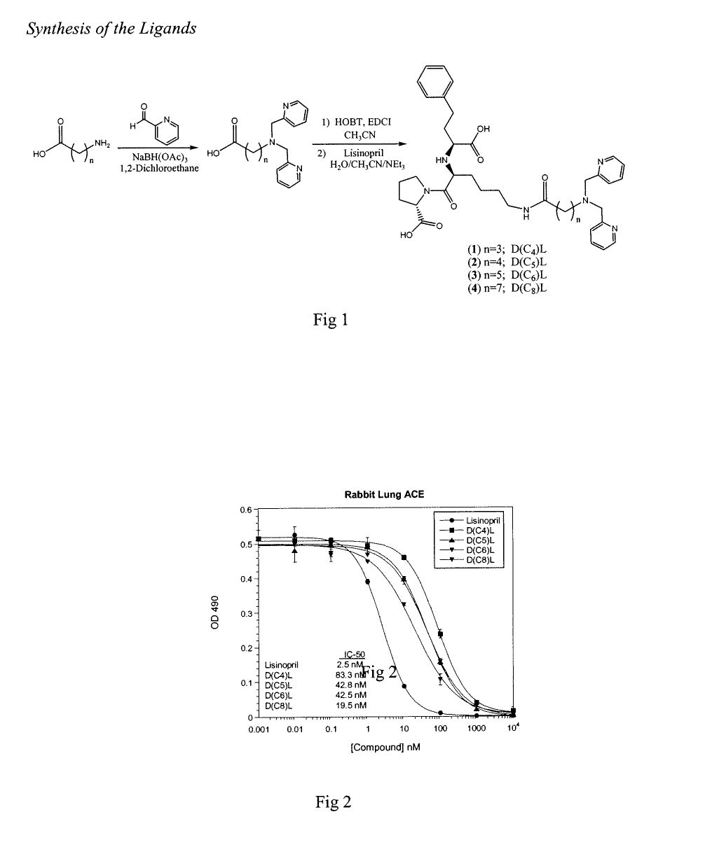

A range of concentrations of each compound was examined for the ability to inhibit ACE cleavage of p-hydroxybenzoyl-glycine L-histidyl-L-leucine using a commercially available in vitro biochemical assay according to manufacturer's specifications (Fujirebio). The source of ACE enzyme chosen for the analysis was purified rabbit lung ACE (Sigrna) at 3.3 mU/sample. Lisinopril was included in each experiment as a positive control. Examples of the data generated by this analysis are shown in FIG. 2. Using rabbit lung ACE; Lisinopril, D(C₄)L, D(C₅)L, D(C₆)L, and D(C₈)L resulted in IC₅₀ values of 2.5 nM, 83.3 nM, and 42.8 nM, 42.5 nM, and 19.5 nM respectively. IC₅₀ values demonstrated that although D(C₈)L (Tissue: 19.5 nM) was not as potent as lisinopril (Tissue: 2.5 nM) it was more potent in comparison to D(C₄)L (Tissue: 83.3 nM). In summary, the in vitro analysis demonstrated that activity increases with increasing number of methylene groups between the dipyridyl group and the core lisinopril moiety.

Similarly, the ability of a conjugate based on a chelating moiety coupled to a small molecule inhibitor of a given peptidase can be evaluated. Table 1 lists an exemplary number of peptidases, along with their substrates. Table 2 lists an exemplary number of small molecule inhibitors of selected peptidases. See, Moskowitz, D. W. Diabetes Technology & Therapeutics (2002) 4(4):519-532 for further discussions on disease states and small molecule inhibitors associated with ACE, in particular.

### In Vivo Analysis

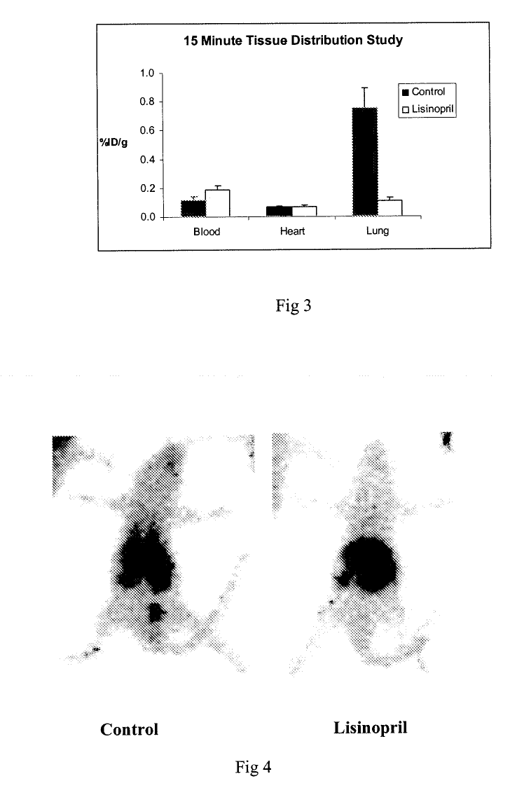

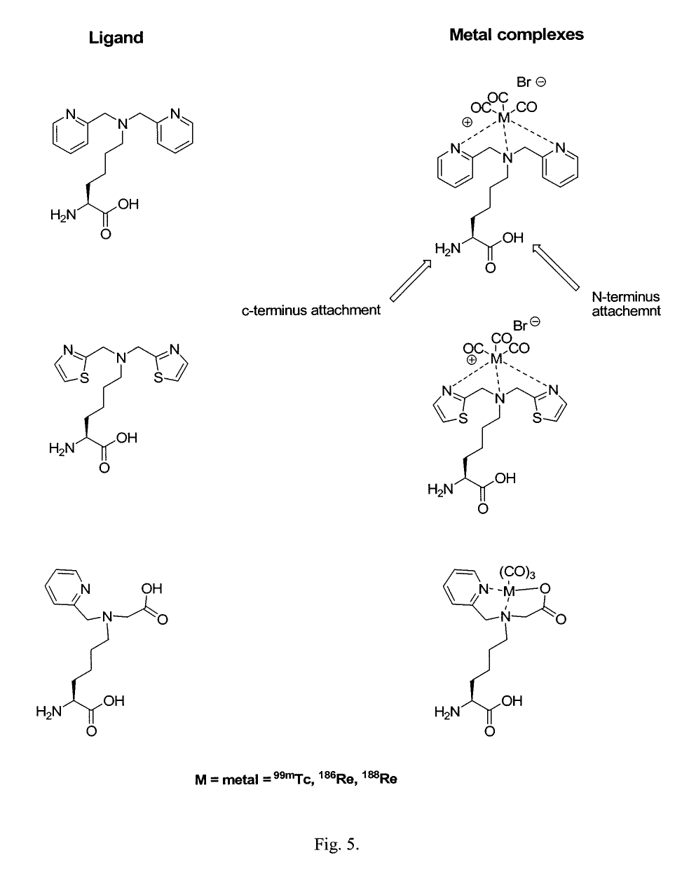

A quantitative analysis of the tissue distribution and clearance of ^{99m}Tc-D(C₅)L was performed in separate groups of normal male Sprague Dawley rats. Animals received 1 mg/kg lisinopril 5 minutes prior to the test compound to block target organ specific uptake and thereby demonstrating the putative mechanism of action in vivo. ^{99m}Tc-D(C₅)L was detected in all tissues examined and decreased steadily over the time course of the experiment. Uptake was observed in the lungs which approached 0.75±0.14% ID/g at 15 minutes post injection (FIG. 3). ^{99m}Tc-D(C₅)L exhibited both renal and hepatobiliary clearance evidenced by the level of compound in the kidneys, liver, and intestines. Pretreatment with 1 mg/kg of lisinopril for 5 minutes before injection of the radiolabeled compound decreased the uptake and retention of compound in the lungs (0.11±10.02% ID/g) suggesting that ^{99m}Tc-D(C₅)L binds specifically to ACE in vivo.

For imaging studies, animals were placed on a gamma camera and baseline planar anterior images consisting of five 1 minute consecutive images were acquired. While a strong signal was detected in the liver and gastrointestinal tract for the compound, ^{99m}Tc-D(C₅)L exhibited lung uptake that was blocked by pretreatment with lisinopril (FIG. 4), corroborating the findings in the tissue distribution studies.

### ACE Colorimetric Assay Protocol

Angiotensin converting enzyme (ACE) activity was determined using the ACE color kit (Fujirebio) according to the manufacturers instructions. ACE acts upon p-hydroxybenzoyl-glycyl-L-histidyl-L-leucine to produce p-hydroxybenzoyl-glycine, which is converted to p-hydroxybenzoic acid by hippuricase. Quinoneimine dye is produced by oxidation and condensation of the p-hydroxybenzoic acid and 4-aminoantipyrine using sodium metaperiodate. The concentration of quinoneimine dye is quantitatively measured at its absorbance maximum of 505 nm. This assay was designed to compare the tissue and plasma specificity of rhenium-labeled ACE inhibitors in an ACE colorimetric assay.

Preparation of rat serum: Blood from normal rats was collected by cardiac puncture with a syringe and 16-gauge needle without anticoagulant and transferred to a 15 ml conical tube. The tube was chilled on ice for 30 min to allow the blood to clot. The clotted blood was removed and the remaining serum was centrifuged at 5,000×g for 10 min at room temperature. The supernatant was recovered and filtered though a 0.22 μm filter.

Preparation of reagents: The ACE color kit was purchased from Fujirebio and the assay was conducted according to the manufacturer's instructions: reconstitute substrate with 5.6 ml of buffer solution, reconstitute blank with 5.6 ml of buffer solution for blank, reconstitute developer with 15.5 ml of stopper solution. The rabbit lung ACE (Sigma A6778) was reconstituted to a concentration of 1 unit/3 ml water.

Assay Method: The optimum concentration of serum and tissue ACE was determined by varying their respective amounts added to the sample or blank tubes according to the following table:

| | | | | | | |
|---|---|---|---|---|---|---|
| 0 | 2.5 | 5 | 10 | 15 | 20 | μL serum |
| | 5 | 10 | 25 | 50 | | μL tissue ACE |

Substrate or blank solution (125 μL) was then added and incubated at 37° C. for 20 min. The developer solution was added and incubated 37° C. for 3 min. The activity of the test compounds was determined by measuring the absorbance at 505 nM on a spectrophotometer. The optimal amount of serum ACE (25 μL) and tissue ACE (3.3 mUnits) was used to determine the specificity of the rhenium-labled ACE inhibitors. Test compounds, including lisinopril and captopril, were prepared (50 μM stock) and serially diluted 10-fold for final concentrations ranging from 1 μM to 0.1 nM (10 μL/assay tube). The assay was conducted as described above.

TABLE 1
Selected Peptidases and Their Substrates

Carboxypeptidase A1
Substrates:
Bz-Gly-Phe
Dns-Gly-Gly-Phe
Dns-Gly-Gly-Trp
Dns-Gly-Phe
Dns-Gly-Trp
Z-Gly-Gly-Leu
Z-Gly-Gly-Phe
Z-Gly-Gly-Val
Carboxypeptidase A2
Substrates:
Z-Gly-Gly-Leu
Z-Gly-Gly-Phe
Z-Gly-Gly-Trp
Z-Gly-Trp
Carboxypeptidase B
Substrates:
Bz-Gly-Arg Bz-Gly-Lys
furylacryloyl-Ala-Arg
Mast Cell Carboxypeptidase A
Carboxypeptidase D
Substrates:
dansyl-Phe-Ala-Arg
Carboxypeptidase E
Carboxypeptidase G, Carboxypeptidase G1,
Carboxypeptidase G2
Substrates:
folic acid
Carboxypeptidase M
Carboxypeptidase N
Carboxypeptidase Y
Substrates:
Z-Gly-Leu
Carboxypeptidase Z
Carboxypeptidase T
Serine Carboxypeptidase A
Substrates:
Bz-Tyr-OEt
dansyl-D-Tyr-Val-NH2
furylacryloyl-Phe-Phe
Z-Glu-Tyr
Z-Phe-Ala
Z-Phe-Leu
Z-Phe-Phe

TABLE 2
Small Molecule Inhibitors of Selected Peptidases

141W94
4-hydroxy-5,6-dihydro-2-pyrone derivative
ABT-378
ABT-538
Ac-Asp-Glu-Val-Asp-H
Ac-DEVD-CHO
Ac-Ile-Glu-Thr-Asp-H
Ac-Leu-Leu-Arg-H
Ac-Leu-Leu-Met-H
Ac-Leu-Leu-Nle-H
Ac-PRLNvs
Ac-Pro-Arg-Leu-AsnVS
Ac-Trp-Glu-His-Asp-H
Ac-Tyr-Val-Ala-Asp-H
Ac-WEHD-CHO
Ac-YVAD-CHO
acetorphan (prodrug)
N-acetyl-aspartyl-glutamyl-valyl-aspartaldehyde
N-acetyl-L-leucyl-L-leucyl-D,L-argininaldehyde
N-acetyl-tryptophanyl-glutamyl-histidinyl-aspartaldehyde
actinonin
active metabolite M8
Ada-Ahx3-L3VS
AdaAhx(3)L(3)VS
AEBSF
AG-1343
AG7088
Agenerase
AGM-1470
aliskiren
ALLM
ALLN
allophenylnorstatine-containing inhibitor
amastatin
[(2S,3R)]-3-amino-2-hydroxy-5-methylhexanoyl]-Val-Val-Asp
2-(5-amino-6-oxo-2-phenyl-pyrimidin-1-yl)-N-[1-hydroxy-3-methyl-1-(5-tert-butyl-
1,3,4-oxadiazol-2-yl)butan-2-yl]acetamide
2-amino-N-[5-(6-dimethylaminopurin-9-yl)-4-hydroxy-2-(hydroxymethyl)oxolan-3-
yl]-3-(4-methoxyphenyl)propanamide
amprenavir
antipain
apstatin
Aptivus
argatroban
arphamenine A
arphamenine B
atazanavir
azidobestatin
bacitracin A
batimastat
BB-2516
BB-94
benzamidine
{1S-benzyl-4R-[1-(1S-carbamoyl-2-phenylethylcarbamoyl)-1S-3-
methylbutylcarbamoyl]-2R-hydroxy-5-phenylpentyl}carbamic acid tert-butyl ester
benzyloxycarbonylphenylalanylarginyldiazomethane
benzylsulfonyl fluoride
bestatin
bestatin analogue SL-387
bestatin, sulfur-containing analogues
BILN2061
BMS-232632
BMS186716
Boc-Ile-Glu-Thr-Asp-H
bortezomib
Brecanavir
butabindide
N-[2-[5-(tert-butyl)-1,3,4-oxadiazol-2-yl]-(IRS)-1-(methylethyl)-2-oxoethyl]-2-(5-
amino-6-oxo-2-phenyl-6H-pyrimidin-1-ly)acetamide
(2S)—N-[(2S,3R)-4-[(3S,4aS,8aS)-3-(tert-butylcarbamoyl)-3,4,4a,5,6,7,8,8a-
octahydro-1H-isoquinolin-2-yl]-3-hydroxy-1-phenyl-butan-2-yl]-2-(quinoline-2-
carbonylamino)butanediamide
Bz-Leu-Leu-Leu-COCHO
BzLLLCOCHO
CA074
calpain inhibitor I
calpain inhibitor II
calpain inhibitor III
candoxatril
candoxatrilat
captopril
N-[(S)-1-carboxy-3-phenylpropyl]-L-Ala-L-Pro
cathepsin L inhibitor Katunuma
CGP-60536
p-chloromercuribenzoate
chymostatin
cilastatin
CKD-731
clasto-lactacystin beta-lactone
CLIK148
CRA-013783
Crixivan
(1S,4R,6S,7Z,14S,18R)-14-cyclopentyloxycarbonylamino-18-[2-(2-
isopropylamino-thiazol-4-yl)-7-methoxyquinolin-4-yloxy]-2,15-dioxo-3,16-
diazatricyclo[14.3.0.04.6]nonadec-7-ene-4-carboxylic acid
D-2-methyl-3-mercaptopropanoyl-L-Pro
D-Phe-Pro-Arg-CH(2)Cl
DANLME
DAPT
darunavir
DCI
DFP
1,3-di-(N-benzyloxycarbonyl-L-leucyl-L-leucyl)aminoacetone
diazoacetyl-D,L-norleucine methyl ester
3,4-dichloroisocoumarin (DCI)
N-[N-(3,5-difluorophenacetyl)-1-alanyl]-S-phenylglycine t-butyl ester
diisopropyl fluorophosphate (DFP)
diisopropyl phosphonofluoridate
(2S)-N-[(2S,4S,5S)-5-[[2-(2,6-dimethylphenoxy)acetyl]amino]-4-hydroxy-1,6-
diphenyl-hexan-2-yl]-3-methyl-2-(2-oxo-1,3-diazinan-1-yl)butanamide
N-[2-[4-(2,2-dimethylpropionyloxy)phenylsulfonylamino] aminoacetic acid
4,6-dioxabicyclo[3.3.0]oct-8-yl [4-[(4-aminophenyl)sulfonyl-(2-
methylpropyl)amino]-3-hydroxy-1-phenyl-butan-2-yl]aminoformate
DPC423
DX-9065a
E-64
E64
E64c
E64d
EDTA
Elaspol
elastatinal
enalapril
enalaprilat
Ep475
EPNP
1,2-epoxy-3(p-nitrophenoxy)propane
EST
N-(2-ethoxy-5-oxo-oxolan-3-yl)-5-isoquinolin-1-ylcarbonylamino-2,6-dioxo-1,7-
diazabicyclo[5.4.0]undecane-8-carboxamide
1-[2-(1-ethoxycarbonyl-3-phenyl-propyl)aminopropanoyl]pyrrolidine-2-carboxylic
acid
ethyl(+)-(2S,3S)-3-[(S)-3-methyl-1-(3-methylbutylcarbamoyl)butylcarbomoyl]-2-
oxiranecarboxylate
N-ethylmaleimide
1-ethylpyrrole-2,5-dione
3-(5-fluoro-3-indolyl)-2-mercapto-(Z)-2-propenoic acid
4-[2-[(4-fluorophenyl)methyl]-6-methyl-5-(5-methyloxazol-3-yl)carbonylamino-4-
oxo-heptanoyl]amino-5-(2-oxopyrrolidin-3-yl)-pent-2-enoate
N-formyl-allo-Ile-Thr-Leu-Val-Pip-Leu-Pip
N-formyl-Val-Thr-Leu-Val-Pip-Leu-Pip
2-[2-(formyl-{allo}-isoleucyl-threonyl-leucyl-valyl)-(hexahydropyradazine-3-
carbonyl)-leucyl]-hexahydropyridazine-3-carboxylic acid
Fortovase
Fosamprenavir (prodrug)
FPRCH2Cl
fumagalone
fumagillin
gamma-secretase inhibitor II
globomycin
GW0385
GW433908
GW433908 (prodrug)
HMBA
HMBSA
1R-[1S,4R,5S]-1-(1-hydroxy-2-methylpropyl)-4-propyl-6-oxa-2-
azabicyclo[3.2.1.]heptane-3,7-dione
(3S,4aS,8aS)-2-[(2R,3R)-2-hydroxy-3-[(3-hydroxy-2-methyl-benzoyl)amino]-4-
phenylsulfanyl-butyl]-N-tert-butyl-3,4,4a,5,6,7,8,8a-octahydro-1H-isoquinoline-3-
carboxamide
(4R)-3-[(2S,3S)-2-hydroxy-3-[[(2R)-2-[(2-isoquinolin-5-yloxyacetyl)amino]-3-
methylsulfanyl-propanoyl]amino]-4-phenyl-butanoyl]-N-tert-butyl-thiazolidine-4-
carboxamide
[1-[[3-hydroxy-4-[(2-methoxycarbonylamino-3,3-dimethyl-butanoyl)amino-[(4-
pyridin-2-ylphenyl)methyl]amino]-1-phenyl-butan-2-yl]carbamoyl]-2,2-dimethyl-
propyl]aminoformate
3-hydroxy-4-[2-[3-hydroxy-6-methyl-4-[3-methyl-2-[3-methyl-2-(3-
methylbutanoylamino)butanoyl]amino-butanoyl]amino-
heptanoyl]aminopropanoylamino]-6-methyl-heptanoic acid
(2S)-1-[(2S,4R)-2-hydroxy-4-[[(1S,2R)-2-hydroxy-2,3-dihydro-1H-inden-1-
yl]carbamoyl]-5-phenyl-pentyl]-4-(pyridin-3-ylmethyl)-N-tert-butyl-piperazine-2-
carboxamide
N-[3-[(1R)-1-[(6R)-2-hydroxy-4-oxo-6-phenethyl-6-propyl-5H-pyran-3-
yl]propyl]phenyl]-5-(trifluoromethyl)pyridine-2-sulfonamide
p-hydroxymercuribenzenesulfonate
p-hydroxymercuribenzoate
IDN-6556
indinavir
invirase
iodoacetamide
iodoacetate
2-iodoacetate
iodotyrostatin
isovaleryl-L-tyrosyl-L-valyl-DL-tyrosinal
N-isovaleryl-tyrosyl-leucyl-tyrosinal
KNI-272
kynostatin-272
L-006235
L-709049
L-735,524
L685458
lactacystin
LAF237
leupeptin
lopinavir
loxistatin
LY-570310
marimastat
MD805
MDL28170
[3-methyl-1-(3-phenyl-2-pyrazin-2-ylcarbonylamino-propanoyl)amino-butyl]boronic
acid
4-methylumbelliferyl p-(NNN-trimethylammonium)cinnamate
4-methylumbelliferyl p-guanidinobenzoate
MG-101
MG-262
MG132
MK-421
MK-422
MK-639
MK0791
MLN-341
MLN519
MQPA
MUGB
MUTMAC
MW167
N-[(S)-2-benzyl-3[(S)(2-amino-4-methylthio)butyl dithio]-1-oxopropyl]-L-
phenylalanine benzyl ester
nelfinavir
NEM
Nip-Leu-Leu-LeuVS-Me
nitrobestatin
NLVS
Norvir
NPGB
NPI-0052
NVP-LAF237
omapatrilat
omuralide
ONO-5046
ONO-6818
OP
ovalicin
6-oxo-5-(3-phenyl-2-sulfanyl-propanoyl)amino-2-thia-7-azabicyclo[5.4.0]undecane-
8-carboxylic acid
6-oxo-6-deoxyfumagillol
oxolan-3-yl [4-[(4-aminophenyl)sulfonyl-(2-methylpropyl)amino]-3-hydroxy-1-phenyl-
butan-2-yl]aminoformate
p-nitrophenyl-p′-guanidinobenzoate
PCMB
PD150606
PD151746
Pefabloc
pepstatin
pepstatin A
1,10-phenanthroline
o-phenanthroline
phenylmethane sulfonylfluoride
2-(phosphonomethyl)pentanedioic acid
phosphoramidon
piperastatin
piperastatin A
PMPA
PMSF
PNU-140690
poststatin
PPACK
pralnacasan
N-(L-3-trans-propylcarbamoyloxirane-2-carbonyl)-L-isoleucyl-L-proline
proteasome inhibitor 3
proteasome inhibitor III
PS-519
PS341
pseudo-iodotyrostatin
pseudo-tyrostatin
PSI-3
PSI-III
puromycin
RB 101(S)
retro-thiorphan [[[(R)-1-(mercaptomethyl)-2-phenylethyl] amino]-3-oxopropanoic
acid] [HSCH2CH(CH2C6H5)NHCOCH2COOH]
ritonavir
RK-805
Ro 31-8959
rupintrivir
ruprintrivir
S-PI
S17092
salinosporamide A
saquinavir
SCH 503034
SCH446211
SCH6
sivelestat
SPP100
SQ14225
SSR69071
statine
TBL(4)K
1,3-thiazol-5-ylmethyl [[3-hydroxy-5-[[3-methyl-2-[[methyl-[(2-propan-2-yl-1,3-
thiazol-4-yl)methyl]carbamoyl]amino]-butanoyl]amino]-1,6-diphenyl-hexan-2-
yl]amino]formate
thiorphan
thiorphan [N-[(S)-2-(mercaptomethyl)-1-oxo-3-phenylpropyl]glycine]
[HSCH2CH(CH2C6H5)CONHC—H2COOH]
tipranavir
TLCK
TMC-95
TMC-95A
TMC-95B
TMC-95C
TMC-95D
TMC114
TNP-470
Tos-LysCH(2)Cl (TLCK)
Tos-PheCH(2)Cl (TPCK)
TPCK
L-trans-epoxysuccinyl-leucylamido(3-methyl)butane
L-trans-epoxysuccinyl-leucylamido(4-guanidino)butane
tyropeptin A
tyropeptin B
tyrostatin
tyrostatin
Ubenimex
UIC-94017
UK-69,578
UK-73,967
UK-79,300
Velcade
vildagliptin
Viracept (nelfinavir mesylate)
VX-740
VX478
VX950
Z-Leu-Leu-leucinal
Z-Leu-Leu-LeuVS
(Z-LL)(2) ketone
Z-Phe-Arg-diazomethane
Z-Val-Phe-H
ZD-8321 (neutrophil elastase inhibitor)
ZL(3)VS
ZL3VS

Other potential inhibitors of any peptidases of interest can be evaluated using a variety of methods. Some exemplary protocols are provided herewith, below.

### Carboxypeptidase A1 and A2

Carboxypeptidase A (CPA) is a pancreatic metallopeptidase hydrolyzing the peptide bond adjacent to the C-terminal end of a polypeptide chain. Carboxypeptidase A1 (CPA1) and carboxypeptidase A2 (CPA2) differ in specificity for peptide substrates: the former (assignable to the traditional A form) shows a wider preference for aliphatic and aromatic residues, whereas the latter is more restrictive for aromatic residues. C-terminal L-amino acids that have aromatic or branched sidechains are preferentially cleaved off the peptide chain.

The determination of reaction velocity is based upon the method of Folk and Schirmer (1963). See, Folk, J., and Schirmer, E. J. Biol. Chem. (1963) 238:3884-94. The rate of hydrolysis of hippuryl-L-phenylalanine (Sigma H6875) is determined by measuring the increase in absorbance at 254 nm. One unit hydrolyzes one micromole of hippuryl-L-phenylalanine per minute at pH 7.5 and 25° C. under the specified conditions.

Substrate

1 mM Hippuryl-L-phenylalanine in 25 mM Tris.HCl, pH 7.5 with 0.5 M sodium chloride.

Enzyme

CPA1 can be purchased through Sigma (C5358). Alternatively, hCPA1 can be purified according to the procedure described by Laethem, et al. Arch Biochem Biophys (1996) 332(1):8-18. hCPA2 can be purified according to the procedure described by Reverter, et al. J. Biol. Chem. (1998) 273(6):3535-41.

Procedure

The stock CPA solution is dissolved in 10% lithium chloride to a final concentration of 1-3 units/mL. The concentration of CPA can be calculated by measuring the absorbance at 278 nm (mg/mL=A₂₇₈×0.515). The substrate is hippuryl-L-phenylalanine (1 mM) in assay buffer (25 mM Tris-HCl, 0.5 M sodium chloride, pH 7.5). Pipette 2.0 mL of substrate into each cuvette and incubate in spectrophotometer at 25° C. for 3-4 minutes to reach temperature equilibration and establish blank rate, if any. Add 0.1 mL of diluted enzyme and record increase in A₂₅₄ for 3-5 minutes. Determine ΔA₂₅₄/minute from the initial linear portion of the curve. The inhibitory activity of test compounds is analyzed by measuring reaction velocity in the presence of concentrations ranging from 1 μM to 0.1 nM.

Calculation

$$Units \text{/} mg = \frac{Δ A 254 \text{/} min}{0.36 * x mg enzyme \text{/} ml reaction mixture}$$

Assay adapted from Worthington Biochem. For more references, see: http ://www.worthington-biochem.com/COA/default.htmL

### Carboxypeptidase B

Carboxypeptidase B (CPB) catalyzes the hydrolysis of the basic amino acids lysine, arginine and ornithine from the C-terminal end of polypeptides. Activity is measured by the spectrophotometric method of Folk and Schirmer (1963) where the reaction velocity is determined by an increase in absorbance at 254 nm resulting from the hydrolysis of hippuryl-L-arginine. One unit causes the hydrolysis of one micromole of hippuryl-L-arginine per minute at 25° C. and pH 7.65 under the specified conditions.

Substrate

1 mM Hippuryl-L-arginine in 25 mM Tris.HCl pH 7.65 containing 0.1 M sodium chloride.

Enzyme

CPB can be purchased through Sigma (C9584). Dilute stock solution with reagent grade water to a concentration of 1-5 units/mL.

Procedure

Pipette 2.9 mL of substrate into cuvette and incubate in spectrophotometer at 25° C. for 3-4 minutes to reach temperature equilibration and establish blank rate, if any. Add 0.1 mL of diluted enzyme and record increase in A₂₅₄ for 3-4 minutes. Determine ΔA₂₅₄/minute from the initial linear portion of the curve. The inhibitory activity of test compounds is analyzed by measuring reaction velocity in the presence of dilutions ranging from 1 μM to 0.1 nM.

Calculation

$$Units \text{/} mg = \frac{Δ A 254 \text{/} min}{0.349 * x mg enzyme \text{/} ml reaction mixture}$$

Assay adapted from Worthington Biochem. For references, see: http://www.worthington-biochem.com/COB/default.htmL

An alternative protocol from Sigma-Aldrich: http://www.sigmaaldrich.com/img/assets/18160/Carboxypeptidase_B.pdf#search=%2 2carboxypeptidase%20b%20assay%22

### Carboxypeptidase D

Carboxypeptidase D (CPD) is a 180-kDa single chain glycoprotein with three homologous carboxypeptidase active site domains and a carboxyl-terminal hydrophobic transmembrane anchor. It cleaves a single amino acid from the C terminus of peptides and proteins and exhibit strict specificity for C-terminal Arginine or Lysine. CPD activity is determined using an endpoint fluorescence assay.

Substrate

The CPD substrate dansyl-L-alanyl-L-arginine is synthesized by reacting dansyl chloride with the dipeptide, alanine-arginine as described previously. See, Proc. Natl. Acad. Sci. U.S.A. (1982) 79:3886-3890; Life Sci. (1982) 31:1841-1844; Methods Enzymol. (1995) 248:663-675.

Enzyme

CPD activity is measured in MCF-7 cell lysates. MCF-7 cells [(10−20)×10⁶] are homogenized with a 21-gauge needle in 0.1 M sodium acetate buffer (pH 5.6). Total cell lysates or subcellular fractions are prepared and Triton X-100 is added to each fraction to give a final concentration of 0.1% (v/v). Samples are stored at −20° C. until further analysis.

Procedure

Ice-cold enzyme sample (60-80 ng of protein/μL in a total volume of 50 μL) is preincubated with 150 μL of 0.1 M sodium acetate buffer (pH 5.6) at 37° C. for 5 min. The assay is initiated by the addition of pre-equilibrated (37° C.) dansyl-L-alanyl-L-arginine substrate (in 50 μL of 0.1 M sodium acetate buffer, pH 5.6). After a 37° C. incubation (6 min for CPD-N and 10 min for CPD), the reaction is terminated by the addition of 150 μL of 1 M citric acid and the sample is placed on ice. The product dansyl-L-alanine is separated from the more hydrophilic substrate, dansyl-L-alanyl-L-arginine, by extraction with chloroform. Fluorescence in the chloroform layer is measured relative to a chloroform blank at 340 nm excitation wavelength and 495 nm emission. Dansyl-L-alanine (Tokyo Chemical Industry America, Portland, Oreg., U.S.A.) is used at various concentrations to construct a standard curve for each assay to correct for the perturbations in extraction efficiency. The inhibitors used are MGTA (DL-2-mercaptomethyl-3-guanidinoethylthiopropanoic acid; Calbiochem, La Jolla, Calif., U.S.A.) and OP (1,10-phenanthroline; Sigma). CP activity is determined as the difference in activity in the presence or absence of 10 μM MGTA. Specific activity SA is calculated as Vₘₐₓ (μmol/min=unit) per mg of protein (i.e. SA=unit/mg of protein). The Km is found to be 63 uM and the Vmax=27 umol/min. The inhibitory activity of test compounds is analyzed at concentrations ranging from 1 μM to 0.1 nM.

Adapted from Biochem J. (2005) 390(Pt 3):665-73 http://www.pubmedcentral.nih.gov/articlerender.fcgi?tool=pubmed&pubmedid=1591 8796

### Carboxypeptidase E

Carboxypeptidase E (CPE) is a processing enzyme that cleaves basic residues from the C-terminus of endoproteolytically cleaved peptide hormones. The enzyme is present exclusively in the Golgi and secretory granules of neural and endocrine cells.

Substrate Dns-Phe-Ala-Arg can be prepared by the method of Fricker Methods Neurosci. (1995) 23:237-250.

Enzyme

Carboxypeptidase E can be purified and isolated by previously established procedures. See, J. Biol. Chem. (1996) 271(8):30619-30624.

Procedure

For the carboxypeptidase assay, 25 μL of enzyme is combined with 50 mM NaAc, pH 5.5 and 200 μM dansyl-Phe-Ala-Arg substrate in a final volume of 250 μL. In addition, tubes contained either 1 mM CoCl₂ or 1 μM guanidinoethylmercaptosuccinic acid (GEMSA). The samples are preincubated with inhibitors for 15 min at 4° C., and then substrate is added and the tubes incubated for 1 h at 37° C. Following incubation for 60 min, 100 μL of 0.5 M HCl and 2 mL of chloroform are added, the tubes mixed, and then centrifuged at 500×g for 2 min. The amount of product is determined by measuring the fluorescence (excitation 350 nm, emission 500 nm) in the chloroform layer. Metallocarboxypeptidase activity is defined as the difference between activity measured in the presence of Co²⁺ (an activator of CPE) and in the presence of GEMSA (an inhibitor of CPE). For these experiments, carboxypeptidase activity is defined as the difference in fluorescence between the tubes containing enzyme and those with only buffer and substrate, and is expressed as the % of the control tube containing enzyme, buffer, and substrate but no divalent ions or inhibitors. The inhibitory activity of test compounds is analyzed at concentrations ranging from 1 μM to 0.1 nM.

Adapted from J. Biol. Chem. (1996) 271(8):30619-24 http://www.jbc.org/cgi/content/full/271/48/30619?ijkey=9114ecbb2b0629a51b4b2c57 82deeec9ed63a931

### Carboxypeptidase G

Carboxypeptidase G is a lysosomal, thiol-dependent protease, which progressively cleaves g-glutamyl pteroyl poly-g-glutamate yielding pteroyl-a-glutamate (folic acid) and free glutamic acid. It is considered highly specific for the g-glutamyl bond, but not for the C-terminal amino acid of the leaving group. (See, J. Biol. Chem. (1967) 242:2933.

Substrate

(+)Amethopterin can be purchased from Sigma-Aldrich (A7019).

Enzyme

Carboxypeptidase G can be purchased from Sigma-Aldrich (C9658). One unit will hydrolyze 1.0 umole glutamic acid from (+)amethopterin per minute at pH 7.3 at 30° C.

Procedure

To 2.8 mL 50 mM Tris HCl Buffer, with 0.1 mM Zinc Chloride, pH 7.3 at 30° C. add 0.1 mL 1.8 mM (+)amethopterin. Mix by inversion and equilibrate to 30° C. Monitor the A320 nm until constant, using a suitably thermostatted spectrophotometer. Then add 0.1 mL enzyme containing 0.3-0.6 unit/mL water. lrnmediately mix by inversion and record the decrease in Δ320 nm/minute using the maximum linear rate for both the Test and the Blank. The inhibitory activity of test compounds is analyzed at concentrations ranging from 1 μM to 0.1 nM.

Calculation

$$Units \text{/} ml enzyme = \frac{( {ΔΔ}_{320 nm} / min Test - Δ {A}_{320 nm} / min Blank ) ( 3 ) ( df )}{( 8.3 ) ( 0.1 )}$$

3=Volume (in milliliters) of assay
df=Dilution factor
8.3=The difference in the millimolar extinction coefficients between the substrate and product at 320 nm.
10.1=Volume (in milliliters) of enzyme used

Adapted from Sigma-Aldrich enzyme assay http://www.sigmaaldrich.com/sigma/enzyme%20assay/c9658enz.pdf

### Carboxypeptidase M

Carboxypeptidase M (CPM) is an extracellular glycosylphosphatidyl-inositol-anchored membrane glycoprotein. This protein is a member of the CPN/E subfamily of zinc metallo-carboxypeptidase. It specifically removes C-terminal basic residues such as lysine and arginine from peptides containing a penultimate alanine. It is believed to play important roles in the control of peptide hormone and growth factor activity on the cell surface, and in the membrane-localized degradation of extracellular proteins (Braz J Med Biol Res 2006 39:211-217).

Substrate Dansyl-Ala-Arg can be synthesized by dansylating the dipeptide Ala-Arg (Methods in Neurosciences: Peptide Technology” (P. M. Conn, ed.), Vol. 6, p. 373. Academic Press, Orlando, Fla., 1991.)

Enzyme

Carboxypeptidase M has been isolated and purified according to the method described by Tan, et al. (Methods Enzymol 1995 248:663-675).

Procedure Add 125 μL of buffer (0.2 M HEPES [4-(2-hydroxyethyl)-1-piperazineethanesulfonic acid], pH 7.0, containing 0.2% (v/v) Triton X-100), 5-50/uL of enzyme sample, 0 or 25 μL of 100 mM MGTA, and 0-70 μL water to give a final volume of 200 μL. For each set of reactions, one enzyme blank (no substrate) and one substrate blank (no enzyme) are prepared. To assure the specificity of the reaction, samples can be preincubated with and without 2-mercaptomethyl-3-guanidinoethylthiopropanoic (MGTA) inhibitor. Samples are preincubated for 5-10 min on ice, and then 50 μL of 1.0 mM Dansyl-Ala-Arg (4.64 mg/10 mL water or dilute 10 mM stock solution 1:10) is added to start the reaction. Samples are incubated at 37° C. for 15 min to 3 hr, depending on activity, and the reaction is stopped by adding 150 μL of the stop solution (1.0 M citric acid adjusted to pH 3.1 with NaOH). Chloroform (1.0 mL) is added to each tube, mixed vigorously for 15 sec to extract the dansyl-Ala product, and then centrifuged at about 800 g for 10 min to separate the phases. The fluorescence in the chloroform layer (bottom layer) is measured relative to a chloroform blank at 340 nm excitation wavelength and 495 nm emission. The inhibitory activity of test compounds is analyzed at concentrations ranging from 1 μM to 0.1 nM.

Calculation

Carboxypeptidase activity is defined as the difference in fluorescence between the uninhibited sample and the sample inhibited with 10 mM MGTA. Fluorescence units (FU) are converted to nanomoles of substrate by constructing a standard curve of FU versus concentration of dansyl-Ala (Sigmna D0125).

Adapted from Methods Enzymol (1995) 248:663-675.

### Carboxypeptidase N

Carboxypeptidase N (CPN) is a plasma zinc metalloprotease, which consists of two enzymatically active small subunits (CPN1) and two large subunits (CPN2) that protect the protein from degradation. CPN cleaves carboxy-terminal arginines and lysines from peptides containing a penultimate alanine found in the bloodstream such as complement anaphylatoxins, kinins, and creatine kinase MM (CK-MM). By removing only one amino acid, CPN has the ability to change peptide activity and receptor binding (Mol Immunol (2004) 40:785-93.

Substrate

Furylacryloyl (FA)-Ala-Lys is commercially available from Sigmna (F5882).

Enzyme

Carboxypeptidase N can be purified according to the method described by Skidgel Methods Enzymol (1995) 248:653-63.

Procedure

Add 0.5 mL of 0.1 M HEPES (pH 7.75) containing 0.5 M NaCl buffer, 0.1 mL of 5 mM FA-Ala-Lys (18.23 mg/10 mL water), and enough water to give a final volume (including sample) of 1.0 mL. The mixture is warmed to 37° C. in a water bath, enzyme sample is added with brief mixing, and then the solution is rapidly transferred to a prewarmed cuvette in a thermostatted (37° C.) chamber of a recording spectrophotometer. The change in absorbance at 336 nm is recorded continuously for about 2-3 min. The inhibitory activity of test compounds is analyzed at concentrations ranging from 1 μM to 0.1 nM.

Adapted from Methods Enzymol (1995) 248:653-63.

### Carboxypeptidase T

Carboxypeptidase T (CPT) was found to be secreted by Thermoactinomyces vulgaris. CPT specificity toward peptide substrates combines the characteristics of carboxypeptidases A and B, that is, the enzyme cleaves off C-terminal neutral, preferably hydrophobic, amino acids, like carboxypeptidase A, and also arginine and lysine residues that bear cationic groups in their side chains.

Enzyme

Carboxypeptidase T can be purified by the method described by Stepanov Methods Enzymol (1995) 248:675-83.

Substrate

Synthesis of Dnp-Ala-Ala-Arg-OH is accomplished through previously described procedures. See, Biokhimiya (1973) 38:790.

Procedure

To 1 mL of 0.5 mM substrate solution in 0.1 M Tris-HCl buffer, pH 7.5, 10-100 μL of the enzyme solution is added. The mixture is incubated for 10-60 min at 37° C., and then 0.2 mL of 50% CH3COOH is added to stop the reaction. The mixture is quantitatively transferred to a microcolumn (plastic cone from an Eppendoff automatic pipette plugged with cotton) that contains 2 mL of SPSephadex C-25, preequilibrated with 1 M CH3COOH. The column is washed with 1 M CH3COOH (two times, 1 mL). The washings are combined, and the A360 of the solution is measured. To calculate Dnp-Ala-Ala-OH concentration, a molar extinction value (e360) of 15,000 is used. One activity unit is equal to the amount of enzyme that hydrolyzes 1˜mol of the substrate in 1 min under the specified conditions. The inhibitory activity of test compounds is analyzed at concentrations ranging from 1 μM to 0.1 nM.

Adpated from Methods Enzymol (1995) 248:675-683.

### Carboxypeptidase Y

Carboxypeptidase Y (CPDY) is a 64 kDa serine carboxypeptidase isolated from Saccharomyces cerevisiae that has been found to catalyze hydrolysis reactions with a large variety of leaving groups, e.g., amino acids, p-nitroaniline, and various alcohols. The assay measures the rate of leucine liberated during the enzymatic hydrolysis of benzyloxycarbonyl-L-phenylalanyl-L-leucine.

Substrate

Benzyloxycarbonyl-L-phenylalanyl-L-leucine can be purchased from Sigma (C1141). Note: 0.5 mL of DMSO (dimethyl solfoxide) is used to dissolve the benzyloxycarbonyl-L-phenylalanyl-L-leucine before mixing with the buffer.

Enzyme

Carboxypeptidase Y is available from Sigma (C3888). Prepare a 1 mg/mL solution of the enzyme, using reagent grade water.

Procedure Add 1.0 mL of 1 mM benzyloxycarbonyl-L-phenylalanyl-L-leucine in 50 mM sodium phosphate, 0.15 M sodium chloride, pH 6.5 substrate solution. Pre-incubate for 10 minutes at 25° C. Start the enzyme reaction by adding 50 μL enzyme. Allow to react at 25° C. for 10 minutes. Add 1.0 mL of the ninhydrin reagent (prepare by mixing 50 mL each of 4% ninhydrin in methyl cellosolve and 0.2 M sodium citrate (pH 5.0)-7.1 mM stannous chloride). Stir for 15 minutes to each of the 10 test tubes. Place all tubes in a boiling water bath for 15 minutes. Remove tubes from bath and cool to below 30° C. Add 5.0 mL of the 50% propanol solution to each of the test tubes and mix well. Read the optical density of all tubes at 570 nm. Leucine is used at various concentrations to construct a standard curve for each assay. The inhibitory activity of test compounds is analyzed at concentrations ranging from 1 μM to 0.1 nM.

Calculation:

$$Units \text{/} ml = \frac{optical density - blank}{slope of standard curve \times 10 minutes \times 0.05}$$

$$Units \text{/} mg = \frac{units \text{/} ml}{mg \text{/} ml sample}$$

Assay adapted from Worthington Biochem. For references, see: http ://www.worthington-biochem.com/COY/default.htmL

### Carboxypeptidase Z

Carboxypeptidase Z (CPZ) is a member of the carboxypeptidase E subfamily of metallocarboxypeptidases. Although these Zn-dependent enzymes have generally been implicated in intra- and extracellular processing of proteins not much is known about the specific substrates of CPZ but it has been shown to cleave C-terminal Arginine and has been implicated in the Wnt signaling pathway. See, Development (2003) 130(21):5103-11.

Substrate

Dansyl-Phe-Ala-Arg can be prepared by the method of Fricker Methods Neurosci. (1995) 23:237-250.

Enzyme

Carboxypeptidase Z cDNA can be stably transfected into AT-20 cells and protein purified by affinity chromatography as previously reported. See, Biochem Biophys Res Comm. (1999) 256:256-8.

Procedure

CPZ activity is assayed using 0.2 mM dansyl-Phe-Ala-Arg in 100 mM, pH 7.4, Tris-Cl buffer in a final buffer volume of 250 μL. After 3 hrs at 37° C., the reaction is terminated with 100 μL of 0.5 M HCl and then 2 mL chloroform are added. After mixing and centrifugation for 2 min at 300×g, the amount of product is determined by measuring the fluorescence in the chloroform phase. To examine the effect of inhibitors, purified CPZ is added to a mixture of buffer, substrate, and inhibitor to give a final concentration of 50 mM Tris-Cl, pH 7.4, 100 uM dansyl-Phe-Ala-Arg and the indicated concentration of inhibitor. The reactions are incubated at 37° C. for 1 hour. Following incubation, 100 μL of 0.5 M HCl and 2 mL of chloroform are added, the tubes mixed, and then centrifuged at 500×g for 2 min. The amount of product is determined by measuring the fluorescence (excitation 350 nm, emission 500 nm) in the chloroform layer. Control reactions without enzyme are performed. Reactions with large amounts of CPE are performed to determine the fluorescence corresponding to complete conversion of substrate into product. The Km values are determined with dansyl-Phe-Ala-Arg and dansyl-Pro-Ala-Arg, using concentrations ranging from 0.025 to 1.6 mM.

Adapted from Biochemical and Biophysical Research Communications (1999) 256:564-568.

### Serine Carboxypeptidase A

Serine carboxypeptidase A also called mammalian cathepsin A, lysosomal carboxypeptidase A and lysosomal protective protein is originally defined as the enzyme which hydrolyzes Z-Glu-Tyr at acidic pH. The enzyme also demonstrates esterase and deamidase activities at neutral pH. Since cathepsin A is able to hydrolyze in vitro a wide spectrum of both synthetic and bioactive peptide hormones such as Z-Phe-Leu, angiotensin II, substance P and endothelin I, it has been suggested that cathepsin A may be implicated in the in vivo metabolism of peptide hormones, although the physiological substrates of cathepsin A are still unknown. The principle of the assay for cathepsin A activity is based on the fluorimetric measurement of N-DNS-Phe liberated enzymatically from the substrate, N-DNS-Phe-Leu, after separation by HPLC.

Enzyme

Mouse kidney homogenates in 0.25 M sucrose centrifuged 100,000×g for 80 min were used as an enzyme source.

Substrate

N-DNS-Phe-Leu was synthesized according to published methods Wiedmeier J. Chromatogr. (1982) 231:410.

Procedure

The reaction mixture contained 50 mM sodium acetate buffer (pH 4.6), 40 μM N-DNS-Phe-Leu, and enzyme plus water in a total reaction volume of 250 μL. Incubation is carried out at 37° C., and the reaction is terminated by heating at 95° C. for 5 min in boiling water. Afier centrifugation, N-DNS-NLeu is added to clear supernatant as the internal standard, and an aliquot of the mixture obtained is subjected to HPLC analysis according to Chikuma, et al. J Chrom B: Biomed Sci and Apps (1999) 728(1):59-65. The peak height of N-DNS-Phe is measured and converted into picomoles from the peak height of N-DNS-NLeu added as an internal standard. One unit of enzyme activity is defined as the amount of enzyme required to convert 1 pmol of the substrate into the corresponding product in 1 min at 37° C. The inhibitory activity of test compounds is analyzed at concentrations ranging from 1 μM to 0.1 nM.

Adapted from J. Chrom. B: Biomed. Sci. and Apps. (1999) 728(1):59-65.

To a round bottom flask containing D(Cₓ)L (1 eq) dissolved in methanol (xx mL) was added [Re(CO)₃(H₂O)₃]Br (1 eq). The reaction was heated to 80° C. and stirred for 4 h. Upon cooling the solvent was removed and the sample was purified by HPLC. The samples were analyzed by ¹H NMR and mass spectroscopy.

Re(CO)₃D(C₄)L (5): Yield=23% (0.4 g). ¹H NMR (CDCl₃, ppm): 8.77 (m, 2H), 7.91 (m 2H), 7.61 (m, 2H), 7.35 (m, 2H), 7.13 (m, 5H), 5.00 (m, 4H), 4.12-2.60 (mm, 11H), 2.26-1.41 (mm, 16H). MS(ESI): m/z 944 (M+H)⁺, m/z 942 (M−H)⁺.

Re(CO)₃D(C₅)L (6): Yield=34% (0.61 g). ¹H NMR (CDCl₃, ppm): 8.77 (m, 2H), 7.91 (m 2H), 7.61 (m, 2H), 7.35 (m, 2H), 7.13 (m, 5H), 3.92 (d, 4H), 3.69-2.65 (mm, 11H), 2.27-1.46 (mm, 18H). MS(ESI): m/z 688 (M+H)⁺, m/z 686 (M−H)⁺.

Re(CO)₃D(C₈)L (7): Yield=55% (0.40 g). ¹H NMR (CDCl₃, ppm): 8.50 (d, 2H), 7.63 (m 2H), 7.50 (m, 2H), 7.13 (m, 8H), 3.85 (d, 4H), 3.69-2.53 (mm, 11H), 2.23-1.22 (mm, 24H). MS(ESI): m/z 730 (M⁺+H)⁺. m/z 728 (M−H)⁺.

General Procedure for ^{99m}Tc(CO)₃D(Cx)L

[^{99m}Tc(CO)₃(H₂O)₃]⁺ was prepared via the Isolink kit using published literature procedures [6]. To test rat plasma stability of the metal complexes, the isolated ^{99m}Tc(CO)₃D(Cx)L were incubated at 37° C. in 1 mL of rat plasma for 5 min, 60 min, and 24 hours. At the desired timepoint an aliquot of the incubation mixtures (400 μL) were removed. Addition of acetonitrile (800 μL) afforded a precipitate which was centrifuged at 15,000 rpm for 5 min. The supernatant was removed and concentrated under a stream of nitrogen. The remaining residue was dissolved in 10% ethanol/Saline and analyzed by HPLC to determine compound stability (FIG. 4).

In vitro ACE activity assay. The ability of test compounds to inhibit ACE activity was determined using the ACEcolor kit from Fujirebio, Inc. according to the manufacturer's instructions. Purified rabbit lung ACE. (3.3 mUnits, Sigrna Chemicals) was incubated for 20 min with the test compound at concentrations of 1 μM to 0.1 nM in a solution of substrate at 37° C. Developer solution was added and the samples were incubated for an additional 5 minutes at 37° C. before reading at 505 nm in a spectrophotometer.

Rat tissue distribution. Tissue distribution studies of ^{99m}Tc(CO)₃D(C₈)L (MIP-1037) were performed in separate groups of male Sprague Dawley rats (n=5/time point). MIP-1037 was administered via the tail vein as a 50 μCi/kg bolus injection (approximately 10 μCi/rat) in a constant volume of 0.1 ml. The animals were euthanized by asphyxiation with carbon dioxide at 10 minutes, 30 minutes, 1 hour, and 2 hours post injection. Tissues (blood, heart, lungs, liver, spleen, kidneys, large and small intestines (with contents), testes, skeletal muscle, and adipose) were dissected, excised, weighed wet, transferred to plastic tubes and counted in an automated γ-counter (LKB Model 1282, Wallac Oy, Finland). Tissue time-radioactivity levels of ^{99m}Tc(CO)₃D(C₈)L (MIP-1037) expressed as % ID/g were determined by converting the decay corrected counts per minute to the percent dose and dividing by the weight of the tissue or organ sample. Aliquots of the injected dose were also measured to convert the counts per minute in each tissue sample to percent injected dose per organ.

Imaging. Six sprague dawley rats were anesthetized with sodium pentobarbital (50 mg/kg, i.p) and randomly assigned to ^{99m}Tc(CO)₃D(C₈)L (MIP-1037) alone or lisinopril/^{99m}Tc(CO)₃D(C₈)L (MIP-1037) treatment groups (n=3/group). All 6 animals were placed on a gamma camera, and baseline planar anterior imaging consisting of five, one-minute consecutive images were acquired using a DSX-LI dual-head γ-camera with a low-energy, all-purpose collimator (SMV America) and Mini Gamma Camera, MGC500 (TeraRecon Inc.) for individual animals. Lisinopril (0.5 mg/kg, i.v.) was administered to animals (n=3) five min prior to ^{99m}Tc(CO)₃D(C₈)L (MIP-1037) administration. After 5 min, 5 mCi/kg ^{99m}Tc(CO)₃D(C₈)L (MIP-1037) was administered i.v. to all animals (n=6), and five one-minute planar anterior images were acquired at 10, 30, and 60 minutes post injection.

Anatomical localization of ^{99m}Tc(CO)₃D(C₈)L (MIP-1037) uptake, utilizing small animal SPECT/CT was also using a X-SPECT small animal scanner with a pinhole collimator (Gamma Medica, Inc., Northridge, Calif.). Rats were injected with ^{99m}Tc(CO)₃D(C₈)L (MIP-1037) alone or with lisinopril (5 minutes before/^{99m}Tc(CO)₃D(C₈)L (MIP-1037)) treatment groups (n=2/group). Rats were anaesthetized with an isofluorane/oxygen mixture. The anesthetized animals were fixed on a special device to guarantee total immobility that is required for later image fusion. The depth of anesthesia was monitored by measuring respiratory frequency using a respiratory belt. Body temperature was controlled by a rectal probe and kept at 37° C. using a thermocoupler and a heated air stream. SPECT data was acquired and reconstructed using the manufacturer's software. Fusion of SPECT and CT data was performed by standard methods.

As illustrated in Table I, the inhibitory activity of each Re-complex, evaluated in vitro against purified rabbit lung ACE, varied directly with the length of the tether (number of methylene spacer units); Re(CO)₃D(C₈)L (MIP-1037); IC₅₀=3 nM), Re(CO)₃D(C₅)L (MIP-1003); IC₅₀=144 nM), and Re(CO)₃D(C₄)L (MIP-1039); IC₅₀=1,146 nM), as compared to lisinopril; IC₅₀=4 nM. The analogue with the seven carbon methylene spacer tether, MIP-1037 exhibited activity that was equivalent to that of the parent molecule, lisinopril.

TABLE I
Inhibitory activity of^{99m}Tc(CO)₃D(Cₓ)L against
purified rabbit lung ACE.

| Compound | n | IC₅₀ (nM) |
|---|---|---|
| MIP-1039 | 3 | 1146 |
| MIP-1003 | 4 | 144 |
| MIP-1037 | 7 | 3 |
| Lisinopril | — | 4 |

Table II shows the rat tissue distribution of ^{99m}Tc(CO)₃D(C₈)L (MIP-1037). The radiotracer was detected at varying levels in all tissues examined and decreased readily over time. Uptake was greatest in the lungs, a tissue with high ACE expression, reaching 15.2% ID/g at 10 minutes post injection, with 3.93% ID/g remaining at 2 hours. Clearance appeared to be primarily via a hepatobillary route as demonstrated by increasing radiolabel in the intestines. Uptake of MIP-1037 was dramatically reduced in the lungs as well as other tissues by coinjection with 0.6 mg/kg non-radiolabeled lisinopril, attesting to specific binding. HPLC analysis of the rat plasma showed that the complex was stable out to 24 hours with no significant decomposition.

TABLE II
Rat tissue distribution of^{99m}Tc(CO)₃D(C₈)L (MIP-1037).

| | 10 minutes | 30 minutes | 1 hour | 2 hour |
|---|---|---|---|---|
| | mean ± SD | mean ± SD | mean ± SD | mean ± SD |
| Blood | 0.15 ± 0.04 | 0.08 ± 0.02 | 0.04 ± 0.02 | 0.04 ± 0.01 |
| | 0.14 ± 0.04 | 0.02 ± 0.01 | 0.01 ± 0.01 | 0.02 ± 0.01 |
| Heart | 0.39 ± 0.06 | 0.21 ± 0.04 | 0.15 ± 0.03 | 0.09 ± 0.02 |
| | 0.07 ± 0.03 | 0.03 ± 0.03 | 0.00 ± 0.01 | 0.03 ± 0.01 |
| Lungs | 15.20 ± 7.36 | 7.05 ± 1.97 | 5.91 ± 1.55 | 3.93 ± 1.17 |
| | 0.17 ± 0.06 | 0.03 ± 0.01 | 0.04 ± 0.06 | 0.02 ± 0.01 |
| Liver | 0.82 ± 0.13 | 0.59 ± 0.18 | 0.34 ± 0.06 | 0.17 ± 0.03 |
| | 2.46 ± 1.15 | 0.29 ± 0.07 | 0.15 ± 0.14 | 0.08 ± 0.01 |
| Spleen | 0.89 ± 0.12 | 0.65 ± 0.18 | 0.01 ± 0.06 | 0.19 ± 0.03 |
| | 0.06 ± 0.01 | 0.01 ± 0.01 | 0.00 ± 0.01 | 0.03 ± 0.01 |
| Kidneys | 1.21 ± 0.27 | 1.33 ± 0.46 | 1.30 ± 0.38 | 0.46 ± 0.14 |
| | 0.54 ± 0.08 | 0.16 ± 0.02 | 0.13 ± 0.02 | 0.08 ± 0.02 |
| Large | 0.18 ± 0.05 | 0.17 ± 0.16 | 0.08 ± 0.02 | 0.10 ± 0.13 |
| Intestine | 0.04 ± 0.02 | 0.02 ± 0.00 | 0.03 ± 0.03 | 0.17 ± 0.32 |
| Small | 1.86 ± 0.77 | 3.41 ± 1.18 | 6.02 ± 0.55 | 6.13 ± 1.36 |
| Intestine | 5.19 ± 2.16 | 6.99 ± 2.39 | 6.67 ± 1.94 | 11.36 ± 1.51 |
| Skeletal | 0.41 ± 0.07 | 0.44 ± 0.16 | 0.36 ± 0.04 | 0.28 ± 0.02 |
| Muscle | 0.08 ± 0.05 | 0.02 ± 0.01 | 0.02 ± 0.01 | 0.04 ± 0.01 |
| Adipose | 0.24 ± 0.09 | 0.29 ± 0.09 | 0.29 ± 0.14 | 0.32 ± 0.06 |
| | 0.07 ± 0.02 | 0.01 ± 0.01 | 0.01 ± 0.02 | 0.06 ± 0.03 |

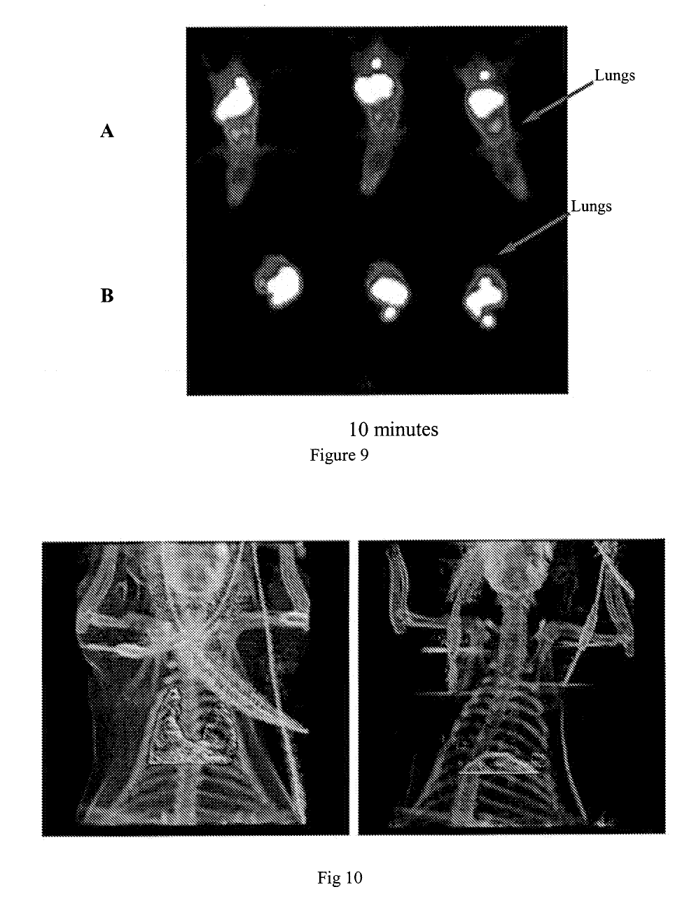

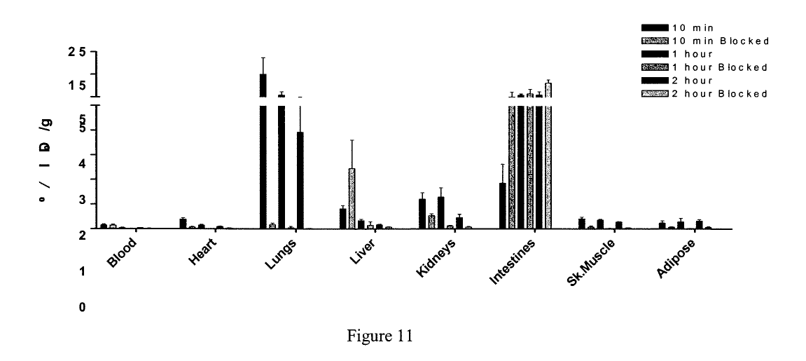

Whole-body imaging was used to determine whether MIP-1037 can be used to non-invasively monitor ACE activity in vivo. As described above, with and without pretreatment with lisinopril rats were used for the in vivo imaging protocol. Regions of interest (ROIs) were drawn over the lung, liver, small bowel, and background (soft tissue) for each animal at each imaging time point. Each ROI was expressed in counts, and the ROIs were normalized to the background at that same time point. FIG. 9 shows in vivo anterior whole-body planar images acquired at 10 minutes after MIP-1037 injection. Initial control images at 10 minutes after injection showed high lung, liver, small bowel, and bladder uptake of the radiotracer that could be blocked by pretreatment with lisinopril.

In addition, small animal SPECT/CT (Gamma Medica, Inc., Northridge, Calif.) imaging studies were performed to define the anatomical localization of the radiotracer. Similar to the whole body planer imaging protocol, rats received MIP-1037 with and without pretreatment of lisinopril as described above. As shown FIG. 10, there was prominent lung activity that was blocked with pretreatment of lisinopril, indicating that a specific binding of MIP-1037 to tissue (lung) ACE in vivo. When images of the pretreatment and control groups were compared, MIP-1037 uptake in the lung was significantly decreased over the 60 minute (all time points) observation period, as were the counts in the ROIs. Radiotracer uptake in the lung nearly disappeared at 60 minutes after injection. In addition, significant decreases in MIP-1037 uptake was also noted in the bladder at 10, 30, and 60 minutes and in the small bowel at 30 and 60 minutes. Liver uptake was transient, and washout from this organ was quite fast and quantitative, with almost all the radioactivity completely eliminated into the intestine at 60 minutes post injection.

The ligands of type D(Cx)L with varying methylene groups were used to form the M(CO)₃⁺ complexes. The most potent compound, M(CO)₃D(C₈)L was tested in vivo using 99m-Tc. The tissue distribution studies showed high uptake in organs containing high ACE expression such as the lungs. Studies with pretreatment of lisinopril showed that the compound was indeed ACE specific. Both planar camera imaging and μSPECT/CT imaging verified the in vivo results. In conclusion, a high affinity Tc-99m labeled ACE inhibitor has been designed with similar potency to lisinopril. Biodistribution, pharmacological blocking studies, and image analysis demonstrates a specific interaction with ACE in vivo. This agent may be useful in monitoring ACE regulation in relevant disease states.

The invention has been described above and illustrated with detailed descriptions of preferred embodiments. It should be apparent to one of ordinary skill in the art, however, that other embodiments fall within the scope of the invention, which should not be limited to the preferred embodiments. Instead the invention should be accorded a scope commensurate with the claims, which follow.

What is cliamed is:

1. A compound comprising a peptidase-binding moiety conjugated to a radiopharmaceutical moiety or an optical imaging moiety.

2. The compound of claim 1 in which the radiopharmaceutical moiety is a radio-imaging moiety, a radio-therapeutic moiety or both.

3. The compound of claim 1 in which the peptidase-binding moiety is selected from exopeptidase or endopeptidases inhibitors.

4. The compound of claim 1 in which the peptidase-binding moiety comprises a carboxypeptidase-binding moiety, which, in turn, is selected from the group consisting of an inhibitor of carboxypeptidase A1, carboxypeptidase A2, carboxypeptidase B, mast cell carboxypeptidase A, carboxypeptidase D, carboxypeptidase E, carboxypeptidase M, carboxypeptidase N, or carboxypeptidase Z.

5. The compound of claim 4 in which the carboxypeptidase-binding moiety comprises an ACE-binding moiety.

6. The compound of claim 5 in which the ACE-binding moiety is selected from the group consisting of alacepril, benazepril, captopril, ceronapril, cilazapril, delapril, enalapril, enalaprilat, fosinopril, imidapril, lisinopril, moexipril, moveltipril, pentopril, perindopril, quinapril, ramipril, rentiapril, spirapril, temocapril, trandolapril, or zofenopril.

7. The compound of claim 1 in which the radio-imaging moiety comprises a radionuclide chelate complex.

8. The compound of claim 7 in which the radionuclide is selected from technetium or rhenium.

9. The compound of claim 8 in which the radionuclide is selected from technetium-99m, rhenium-186, or rhenium-188.

10. The compound of claim 1 in which the radio-imaging moiety comprises a (technetium-99m)Tc(CO)₃ or (rhenium-186/188)Re(CO)₃ chelate complex.

11. The compound of claim 5 in which the ACE-binding moiety inhibits tissue ACE to a greater extent than serum ACE.

12. The compound of claim 5 whose IC₅₀ inhibition of ACE is less than 20 nM.

13. The compound of claim 1 in which the peptidase-binding moiety and the radio-imaging moiety are conjugated via an amide, ester, amine, or ether linkage.

14. A method of imaging one or more organs or tissues or both of a mammal comprising administering to a mammal an effective amount of a compound comprising a peptidase-binding moiety conjugated to a radio-imaging moiety or an optical imaging moiety and obtaining an image of one or more organs or tissues or both of the mammal.

15. The method of claim 14 in which the compound is administered intravenously.

16. The method of claim 14 in which the compound is selected from the group consisting of cold rhenium-labeled or technetium-99m-labeled D(C4)L (1), D(C5)L (2), D(C6)L (3), or D(C8)L (4).

17. The method of claim 14 in which the one or more organs or tissues or both includes lung tissue.

18. The method of claim 14 in which the one or more organs or tissues or both includes kidney tissue.

19. The method of claim 14 in which the one or more organs or tissues or both includes heart tissue.

20. The method of claim 14 in which the one or more organs or tissues or both includes tumor tissue.

21. The method of claim 14 in which the one or more organs or tissues or both includes a vulnerable plaque condition.

22. The method of claim 14 in which the one or more organs or tissues or both includes an atherosclerotic condition.

23. The method of claim 14 in which the one or more organs or tissues or both includes an inflammatory condition.

24. A kit comprising: (i) compound comprising a peptidase-binding moiety conjugated to a metal chelating moiety, and (ii) radionuclide.

25. The kit of claim 24 in which the radionuclide is selected from technetium-99m, rhenium-186, rhenium-188 or combinations thereof.

26. A method of staging a pathological condition associated with one or more organs or tissues or both of a mammal comprising: (i) administering to a mammal an effective amount of a compound comprising a peptidase-binding moiety conjugated to a radio-imaging moiety, (ii) obtaining an image of the one or more organs or tissues or both of said mammal; (iii) determining from said image the amount of peptidase which is present in the one or more organs or tissues or both of said mammal, and (iv) utilizing the amount determined and a control amount to arrive at a stage of the pathological condition.

27. The method of claim 26 in which the pathological condition is selected from the group consisting of heart failure, cardiomyopathy, lung disease, kidney dysfunction, renal failure, inflammation, atherosclerosis, vulnerable arterial plaques or neoplasm.

28. A method of monitoring a mammal's response to therapy for a pathological condition associated with one or more organs or tissues or both of the mammal comprising (i) administering to a mammal an effective amount of a compound comprising a peptidase-binding moiety conjugated to a radio-imaging moiety, (ii) obtaining an image of the one or more organs or tissues or both of the mammal, (iii) determining from said image the amount of peptidase which is present in the one or more organs or tissues or both of the mammal, and (iv) utilizing the amount determined and a control amount to gauge the mammal's response, if any, to a therapy.

29. The method of claim 26 in which the control amount is obtained from an amount found in a group of normals.

30. The method of claim 26 in which the control amount is obtained from a baseline amount found in the one or more organs of said mammal.

31. The method of claim 28 in which the control amount is obtained from an amount found in a group of normals.

32. The method of claim 28 in which the control amount is obtained from a baseline amount found in the one or more organs of the mammal.

33. A method of quantifying expression of a peptidase in one or more organs or tissues or both of a mammal comprising administering to a mammal an effective amount of a compound including a peptidase-binding moiety conjugated to a radio-imaging moiety, obtaining an image of the one or more organs or tissues or both of the mammal; quantifying from the image and a series of standard images an amount of expression of the peptidase in the one or more organs or tissues or both of the mammal.

34. A method of subjecting a mammal in need thereof to radiotherapeutic treatment comprising administering to a mammal an effective amount of a compound comprising a peptidase-binding moiety conjugated to a radiotherapeutic moiety.

35. The method of claim 34 in which the compound is administered intravenously.

36. The method of claim 34 in which the mammal is suffering from a neoplastic condition.

37. A compound of the following formula:
(PBM)ₙ-(LIN)-(CHE)ₘ
wherein
PBM comprises a peptidase binding moiety,
n is 1, 2 or 3,
LIN is a covalent bond, —CH₂—, —NH—, or a linear or branched chain that is 2-20 carbon atoms in length, and optionally bonded to or within the chain are 1-6 heteroatoms including amino, oxygen, sulfur, carbonyl, urea, or amide, aromatic rings, cyclic aliphatic rings, heteroaromatic rings, or heterocyclic aliphatic rings, and which covalently links the one or more PBMs with the one or more CHEs;
CHE comprises a chelating moiety that can be a monodentate, bidentate or polydentate ligand capable of binding a radionuclide and
m is 1, 2 or 3.

38. The compound of claim 37, wherein the peptidase binding moiety is an inhibitor of carboxypeptidase A1, carboxypeptidase A2, carboxypeptidase B, mast cell carboxypeptidase A, carboxypeptidase D, carboxypeptidase E, carboxypeptidase M, carboxypeptidase N, or carboxypeptidase Z.

39. The compound of claim 37, wherein the peptidase binding moiety is alacepril, benazepril, captopril, ceronapril, cilazapril, delapril, enalapril, enalaprilat, fosinopril, imidapril, lisinopril, moexipril, moveltipril, pentopril, perindopril, quinapril, ramipril, rentiapril, spirapril, temocapril, trandolapril, or zofenopril.

40. The compound of claim 37, wherein the linker is a 2-15 atom chain, wherein in 1-6 atoms of the chain are amino, oxygen, sulfur, carbonyl, urea or amide and the rest of the atoms of the chain are carbon.

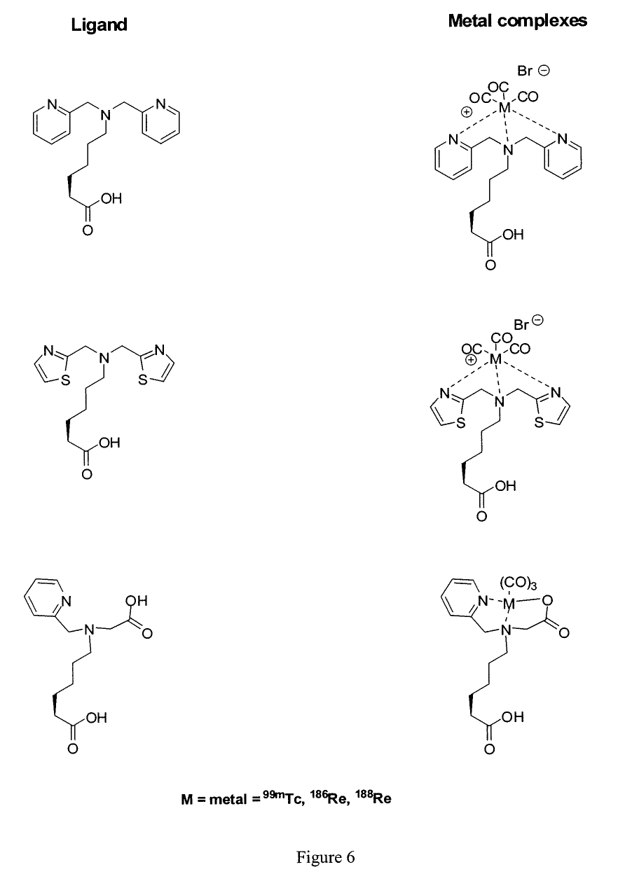

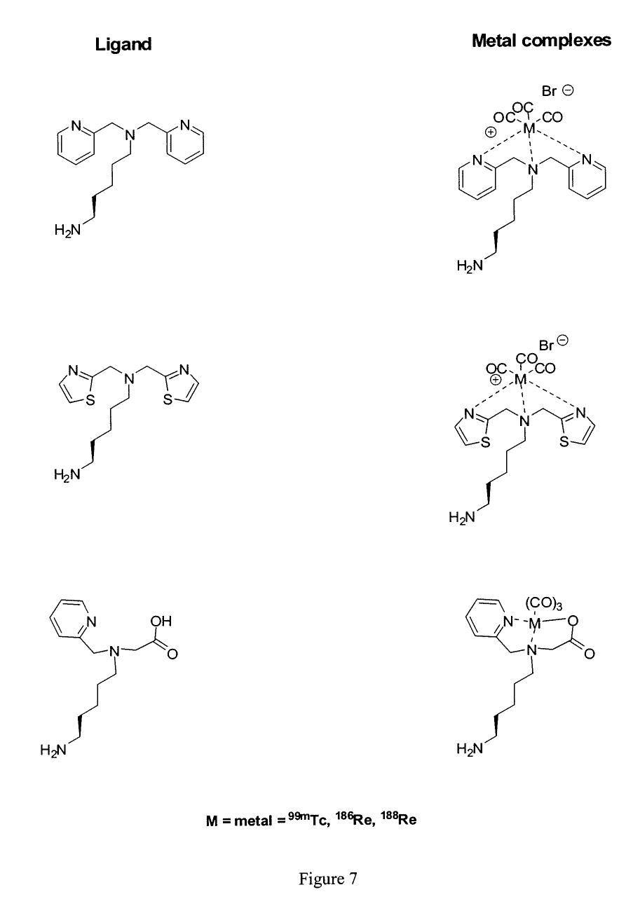

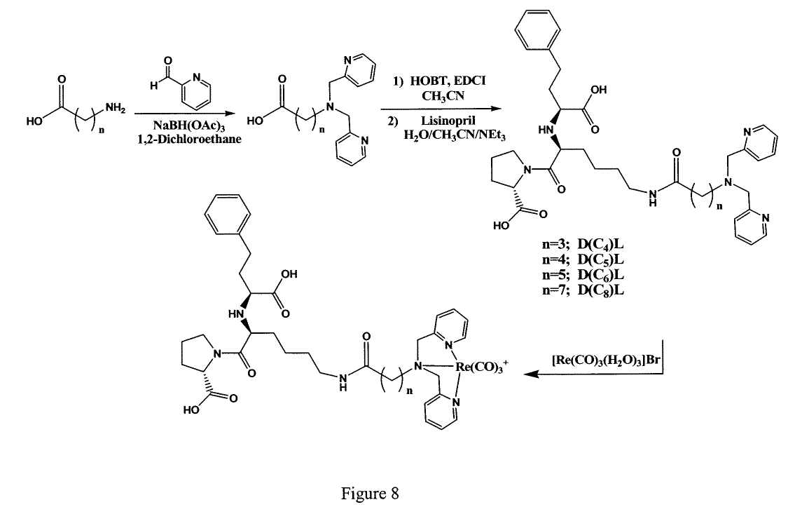

41. The compound of claim 40, wherein the linker comprises a lysine or a lysine analogue, such as the lysine analogues shown in FIGS. 6 or FIGS. 7.

42. The compound of claim 37, wherein the radionuclide is Tc or Re.

43. The compound of claim 37, wherein the CHE moiety is pyridylmethylene amine, quinolinemethylene amine, isoquinoline amine, pyridine-2-ylmethylamino acetic acid, isoquinolin-3-yhnethylamino acetic acid, thiazol-2-ylmethyl amine, and thiazol-2-ylmethylamino acetic acid or chelators of the following structures, which are shown as being bound to Tc: 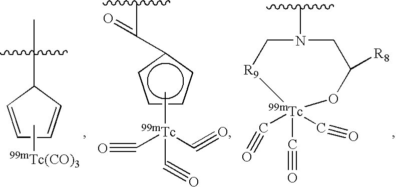
R₈ is selected from the group O, H, OH, alkoxy, or O-alkyl;
R₉ is a pharmaceutically acceptable heterocycle, such as a 5 or 6 membered ring with 1-2 nitrogen, oxygen or sulfur atoms, 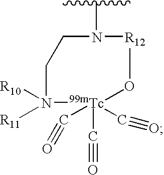
R₈ is selected from the group O, H, OH, alkoxy, or O-alkyl;
R₉ is a pharmaceutically acceptable heterocycle, such as a 5 or 6 membered ring with 1-2 nitrogen, oxygen or sulfur atoms,
R₁₀ and R₁₁ are each independently hydrogen, alkyl, or substituted alkyl;
R₁₂ is selected from the group of aryl, alkyl, or heterocycle; 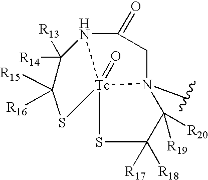
R₁₃, R₁₄, R₁₅, R₁₆, R₁₇, R₁₈, R₁₉, R₂₀ are independently Hydrogen or methyl [chemical structure]
